(19) United States
(12) Patent Application Publication
(10) Pub. No.: US 2020/0013508 A1
(43) Pub. Date: Jan. 9, 2020

(54) SYMPTOM STANDARDIZATION MATCHING SYSTEM

(71) Applicant: PARTNERS & CO Inc., Seongnam-si (KR)

(72) Inventor: Seichul CHUN, Paju-si (KR)

(73) Assignee: PARTNERS & CO Inc., Seongnam-si (KR)

(21) Appl. No.: 16/056,539

(22) Filed: Aug. 7, 2018

(30) Foreign Application Priority Data
Jul. 4, 2018 (KR) 10-2018-0077770

(51) Int. Cl. G16H 50/20; G06F 17/30

(52) U.S. Cl. CPC G16H 50/20; G06F 17/30533

(57) ABSTRACT

A symptom standardization matching system may include a communication module configured to receive a symptom from a user terminal, an extraction module configured to extract a symptom expression from a symptom table with respect to the symptom received through the communication module, and a matching module configured to match a corresponding standard symptom to the symptom expression extracted by the extraction module through a symptom standardization model and to match at least one related service of a candidate service group corresponding to a standard symptom matched through a symptom service matching model to the user terminal.

### CROSS REFERENCE TO RELATED APPLICATION

The present application claims the benefit of Korean Patent Application No. 10-2018-0077770 filed in the Korean Intellectual Property Office on Jul. 4, 2018, the entire contents of which are incorporated herein by reference.

### BACKGROUND OF THE INVENTION

### 1. Technical Field

The present invention relates to a symptom standardization matching system and is the results of Industry and Academic Research Cooperation Business of Seoul supported by Seoul Business Agency (SBA) of Seoul.

### 2. Description of the Related Art

With the development of the Internet, telemedicine services using the Internet are developed, and thus online medical services such as diagnosis, treatment and consultation are actively performed.

As a kind of such medical services, there is a telemedicine system for allowing a patient to autonomously diagnose a degree of risk of a disease through given survey and to send corresponding data to the telemedicine system and a medical specialist to diagnose the disease of the patient based on such data. However, such a simple system has a limit to accurate symptom diagnosis.

### SUMMARY OF THE INVENTION

An embodiment of the present invention is to provide a symptom standardization matching system for providing a patient with more accurate customized services through an algorithm capable of improving the accuracy of a symptom of the patient.

A symptom standardization matching system according to an embodiment of the present invention may include a communication module configured to receive a symptom from a user terminal, an extraction module configured to extract a symptom expression from a symptom table with respect to the symptom received through the communication module, and a matching module configured to match a corresponding standard symptom to the symptom expression extracted by the extraction module through a symptom standardization model and to match at least one related service of a candidate service group corresponding to a standard symptom matched through a symptom service matching model to the user terminal.

Furthermore, the symptom standardization matching system may further include a review module configured to extract the symptom expression in a review using the symptom table through the extraction module in order to select the symptom expression in the review when the review into the at least one related service received from the user terminal is registered.

Furthermore, the symptom standardization matching system may further include a verification improvement module configured to verify and improve the symptom standardization model and the symptom service matching model based on the symptom expression extracted through the review module and the extraction module.

Furthermore, the symptom standardization matching system may further include a database configured to store the symptom table, wherein the symptom table comprises a standard symptom, code corresponding to the standard symptom, a similar symptom, and symptom expression information.

Furthermore, the standard symptom is divided into two or more and stored in the symptom table, a corresponding code may be assigned to each standard symptom, and a similar symptom determined to be a symptom similar to the standard symptom may be previously set and matched to the code. The matching module may set a symptom expression for at least one symptom based on information received from the user terminal, may match a standard symptom corresponding to the set symptom expression, and may check code corresponding to the matched standard symptom.

Furthermore, the similar symptom may correspond to that a predetermined number of pieces of information or more set as the symptom expression matched to the standard symptom are included in common with the standard symptom.

Furthermore, the verification improvement module may make an additional question to the user terminal, may add and update the symptom expression by taking into consideration an answer received from the user terminal, and may set the standard symptom or similar symptom again based on the updated information.

A service matching system for a symptom according to an embodiment of the present invention may include a user terminal, a provider terminal configured to provide the user terminal with at least one related service corresponding to a symptom, and a symptom standardization matching system connected to the user terminal and the provider terminal and configured to control the at least one related service.

### BRIEF DESCRIPTION OF THE DRAWINGS

Detailed description of each figure is provided so that the figures cited in the detailed description of the present invention can be understood more sufficiently.

FIG. 1 shows the entire system according to an embodiment of the present invention.

FIG. 2 is a block diagram specifically showing a symptom standardization matching system of FIG. 1.

FIG. 3 is a flowchart for illustrating a symptom standardization matching method according to an embodiment of the present invention.

FIG. 4 is a flowchart for illustrating a detailed improvement method of the symptom standardization matching method according to an embodiment of the present invention.

FIG. 5 is a diagram showing an execution service of the symptom standardization matching system of FIG. 1.

FIG. 6 is a diagram showing a symptom table according to an embodiment of the present invention.

### DETAILED DESCRIPTION

The above and other aspects, features and advantages of the invention will become apparent from the following description of the following embodiments given in conjunction with the accompanying drawings. However, the inventive concept is not limited to the embodiments disclosed below, but may be implemented in various forms. The embodiments of the inventive concept is provided to make the disclosure of the inventive concept complete and fully inform those skilled in the art to which the inventive concept pertains of the scope of the inventive concept.

Although not defined, all the terms (including technical or scientific terms) used herein may have the same meanings that are generally accepted by the common technologies in the field to which the inventive concept pertains. The terms defined by the general dictionaries may be construed to having the same meanings as those meant in the related technologies and/or the disclosure of the application, and will neither become conceptual nor be construed to be excessively formal even though not clearly defined herein.

The terms used herein are provided to describe the embodiments but not to limit the inventive concept. In the specification, the singular forms include plural forms unless particularly mentioned. The expressions ‘include’ and/or its various conjugated forms, such as ‘including’, which are used in the specification do not exclude existence or addition of one or more compositions, substances, elements, steps, operations, and/or devices. In the specification, the term ‘and/or’ represents enumerated configurations or various combinations thereof.

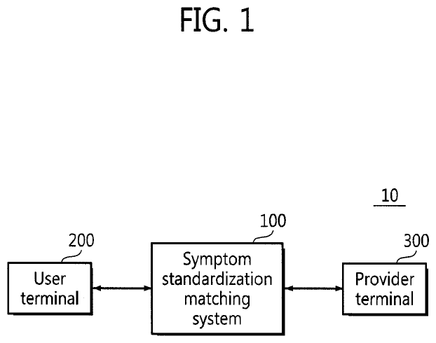

FIG. 1 shows the entire system 10 according to an embodiment of the present invention. Referring to FIG. 1, the entire system 10 includes a symptom standardization matching system 100, a user terminal 200 and a provider terminal 300.

The symptom standardization matching system 100, the user terminal 200 and the provider terminal 300 may be connected over a network.

The symptom standardization matching system 100 may correspond to the contact point of an online user and provider over a network. The system may provide customized services, such as member subscription, the provision of a variety of types of information, user physical condition check using a medical examination, the service matching and recommendation of a user and a provider, counsel with a provider that is an expert for a user's symptom, reservation, and product purchase.

Furthermore, the symptom standardization matching system 100 may manage user experiences regarding expectations and effects for services and programs provided by providers and reviews written by users.

Furthermore, the symptom standardization matching system 100 may store, analyze and personalize a user's activity history, and may recommend similar contents for a field of interest.

Furthermore, the symptoms standardization matching system 100 may record experiences regarding a service, product or tool provided by a provider, may perform reputation management, and may integrate and manage a variety of types of data (e.g., writings, figures, voice, images and links) regarding a service, tool, program, etc. provided by a provider.

In accordance with the symptom standardization matching system 100, a user can reduce the time and cost in a complicated and inefficient conventional purchase process and can perform all of choices and a purchase process in one app or web more simply.

In accordance with the symptom standardization matching system 100, a provider can discover a new customer, can be induced to reduce a marketing cost, and can provide a solution capable of integrating a variety of types of data simply and conveniently.

The user terminal 200 may correspond to an IoT terminal including a portable device, such as a smartphone or a tablet PC. The user terminal may transmit symptom information, user personal information, past symptom history information, and life pattern information, input by the user of the user terminal 200, to the symptom standardization matching system 100. In this case, the user personal information may correspond to the age, sex and occupation group of the user. The past symptom history information may correspond to information about the symptoms of a disease suffered by the user in the past. The user life pattern information may include information about exercise or eating habits performed by the user periodically or repeatedly.

The provider terminal 300 provides the user terminal 200 with customized services, a program or a product through the symptom standardization matching system 100. The provider terminal may correspond to an IoT terminal including a portable device, such as a smartphone or a tablet PC.

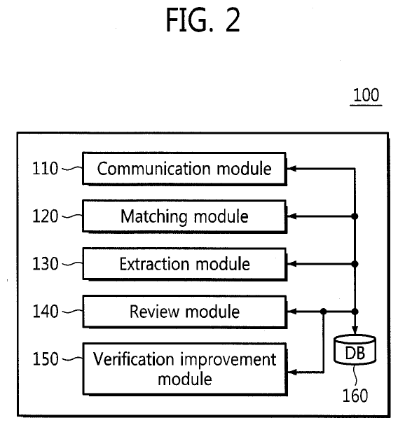

FIG. 2 is a block diagram specifically showing the symptom standardization matching system 100 of FIG. 1. The symptom standardization matching system 100 includes a communication module 110, a matching module 120, an extraction module 130, a review module 140, a verification improvement module 150 and a database (DB) 160. The matching system 100 may be hardware such as a microprocessor or an electronic circuit or software such as a program or firmware executed by the hardware, and also may be implemented by combining the hardware and the software.

Referring to FIG. 2, the communication module 110 performs a function of exchanging a variety of types of data with the user terminal 200 and the provider terminal 300. For example, when the user of the user terminal 200 complains of a symptom using a natural language, information about the symptom may be received through the communication module 110.

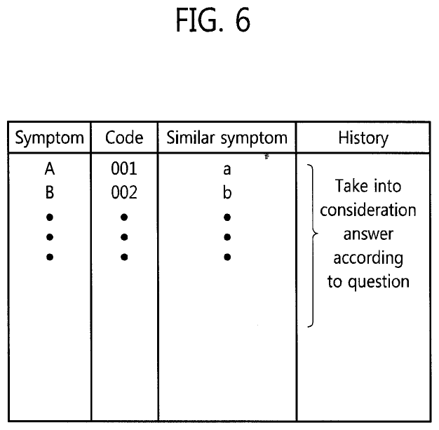

The extraction module 130 may extract a symptom expression using a symptom table stored in the DB 160 with respect to information about an input symptom. The symptom table and the symptom expression are described more specifically with reference to FIG. 6.

The matching module 120 may match a corresponding standard symptom to an extracted symptom expression using a symptom standardization model. For example, referring to FIG. 6, if an extracted symptom expression corresponds to code 001, a corresponding standard symptom A or B may be matched to the code 001.

Furthermore, the matching module 120 may match at least one related service of a candidate service group, corresponding to a standard symptom matched through a symptom service matching model, to the user terminal 200.

When the user terminal 200 registers a received service review, the review module 140 may select a symptom expression in the registered review and enable the symptom expression in the review to be extracted using the symptom table through the extraction module 130.

The verification improvement module 150 may improve the symptom standardization model and the symptom service matching model. The symptom standardization model may be updated based on a user's basic personal information and the user's past history registration input information. The symptom standardization model and the symptom service matching model may be updated through a symptom expression extracted based on a review.

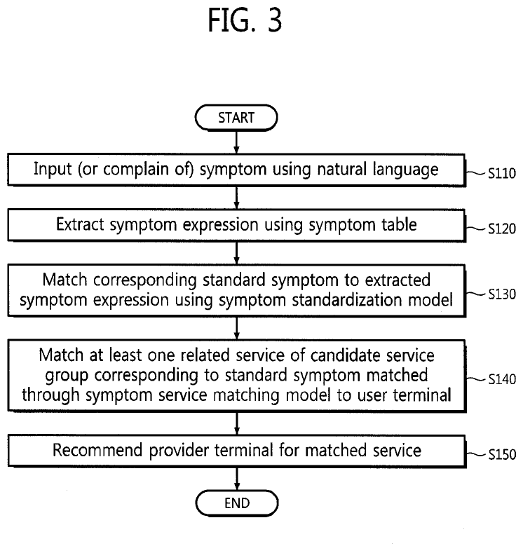

FIG. 3 is a flowchart for illustrating a symptom standardization matching method according to an embodiment of the present invention. The symptom standardization matching method may be performed by the symptom standardization matching system 100 of FIG. 1.

Referring to FIG. 3, when the user of the user terminal complains of a symptom using a natural language, information about the symptom is input to the symptom standardization matching system 100 (S110).

A symptom expression is extracted using the symptom table stored in the DB with respect to the input information about the symptom (S120). The symptom table and the symptom expression have been described more specifically with reference to FIG. 6.

Next, a corresponding standard symptom is matched to the extracted symptom expression using the symptom standardization model (S130). For example, referring to FIG. 6, if the extracted symptom expression corresponds to code 001, a corresponding standard symptom A or B may be matched to the code 001.

At least one related service of a candidate service group corresponding to the standard symptom matched through the symptom service matching model is matched to the user terminal 200 (S140). Accordingly, an optimized service can be matched to the user of the user terminal 200 who has input the symptom.

The provider terminal 300 for the matched service is recommended to the user terminal 200, so they can be connected (S150).

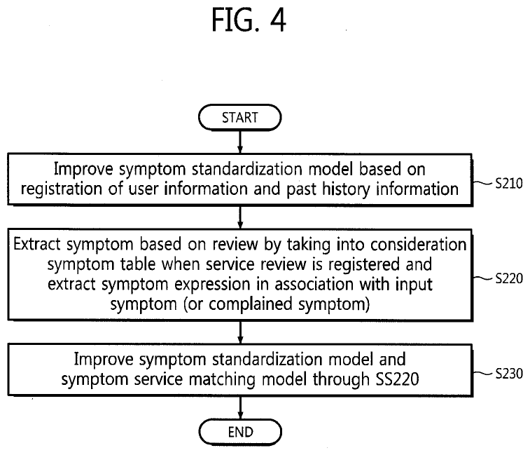

FIG. 4 is a flowchart for illustrating a detailed improvement method of the symptom standardization matching method according to an embodiment of the present invention. The improvement method of FIG. 4 may be performed by the symptom standardization matching system 100 of FIG. 1.

Referring to FIG. 4, the symptom standardization model is improved by registering user information and past history information (S210). When a service review is registered, a symptom based on the review is extracted by taking into consideration the symptom table. A symptom expression is extracted in association with the input symptom (or complained symptom) (S220).

Thereafter, the symptom standardization model and the symptom service matching model are updated or improved through step S220 (S230). Accordingly, symptom determination accuracy and service matching accuracy can be improved.

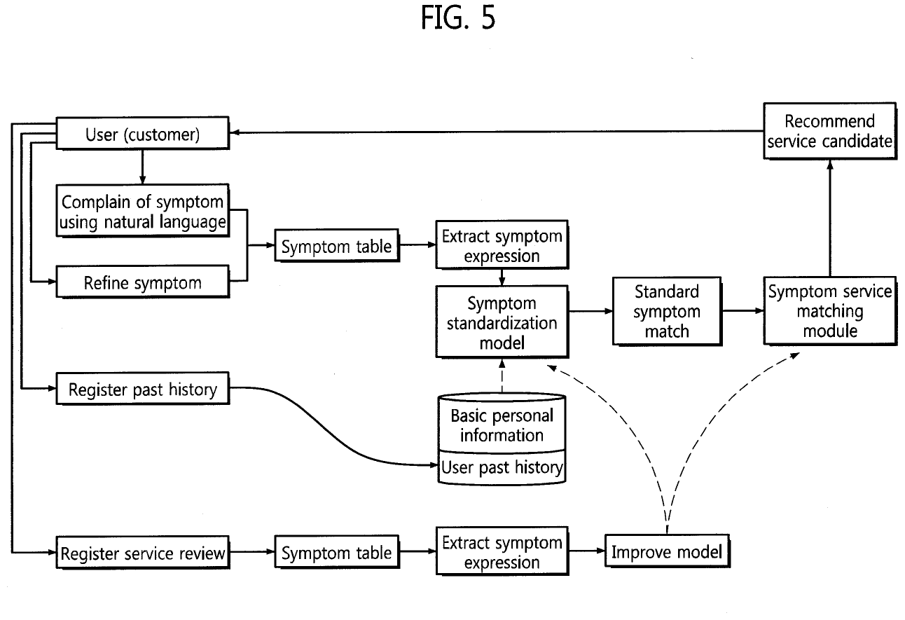

FIG. 5 is a diagram showing an execution service of the symptom standardization matching system 100 of FIG. 1.

Referring to FIG. 5, a user (or customer) complains of a symptom in a natural language. When the symptom is received through the communication module, that is, an input unit, a symptom expression is extracted using the symptom table stored in the DB. Furthermore, the user (or customer) may refine the symptom, if necessary. A symptom expression may be extracted using the symptom table by additionally receiving the refined symptom through the communication module, that is, an input unit, and taking into consideration the refined symptom.

The symptom expression may be extracted through the symptom standardization model. A standard symptom may be matched through the symptom service matching model. In this case, the symptom standardization model may be updated based on the user's basic personal information and past history registration input information. More accurate information may be provided through updates.

Furthermore, the user (or customer) may register a received service review and extracts a symptom expression in the review using the symptom table. Accordingly, the symptom standardization model and symptom service matching model for the symptom corresponding to the user can be improved by taking into consideration the extracted symptom expression [symbol] the user.

FIG. 6 is a diagram showing a symptom table according to an embodiment of the present invention. The symptom table includes symptom, codes according to the symptoms, similar symptoms, and information about the history of the symptoms. In this case, the history of the symptom may include information about a symptom expression.

For example, a symptom may be divided in plural number and stored in the symptom table. Corresponding code 001 may be assigned to a symptom (or standard symptom) A. A similar symptom classified as a symptom similar to the symptom A may have been matched to the symptom A.

The symptom standardization matching system 100 may set the history of at least one symptom or at least one symptom expression based on information complained by the user of the user terminal or input information from the user terminal, may match a corresponding symptom (or standard symptom), and may check a corresponding code.

Not only the symptom (or standard symptom), but a similar symptom classified as being similar to the symptom (or standard symptom) is previously set and matched to the code. In this case, a criterion for the similarity may mean that a predetermined number of pieces of information or more set as the history of a symptom or a symptom expression are included in common.

The history of the symptom or the symptom expression may be added and updated by taking into consideration a corresponding answer after additionally asking the user terminal if necessary. A symptom (or standard symptom) or a similar symptom is set again based on the updated information, thereby being capable of improving accuracy.

The symptom standardization matching system and method according to the embodiments of the present invention have an effect in that more accurate customized services can be provided to a patient through an algorithm for improving the accuracy of a symptom of the patient.

According to an embodiment of the inventive concept, even only by attaching the enhancement member to the apparatus for generating the plasma, the effect of using the plasma may be maximized through enhancing the action caused by the plasma. In particular, various therapeutic effects including the plasma sterilization effect may be greatly improved by appropriately selecting the enhancement material provided in the plasma enhancement member.

Although the inventive concept has been described through the embodiment, the embodiment is provided simply to describe the spirit of the inventive concept and the inventive concept is not limited thereto. It is noted that those skilled in the art may variously modify the embodiment. The scope of the inventive concept is determined only through analysis of the claims.

What is claimed is:

1. A symptom standardization matching system, comprising:
a communication module configured to receive a symptom from a user terminal;
an extraction module configured to extract a symptom expression from a symptom table with respect to the symptom received through the communication module; and
a matching module configured to match a corresponding standard symptom to the symptom expression extracted by the extraction module through a symptom standardization model and to match at least one related service of a candidate service group corresponding to a standard symptom matched through a symptom service matching model to the user terminal.

2. The symptom standardization matching system of claim 1, further comprising a review module configured to extract the symptom expression in a review using the symptom table through the extraction module in order to select the symptom expression in the review when the review into the at least one related service received from the user terminal is registered.

3. The symptom standardization matching system of claim 2, further comprising a verification improvement module configured to verify and improve the symptom standardization model and the symptom service matching model based on the symptom expression extracted through the review module and the extraction module.

4. The symptom standardization matching system of claim 3, further comprising a database configured to store the symptom table, wherein the symptom table comprises a standard symptom, code corresponding to the standard symptom, a similar symptom, and symptom expression information.

5. The symptom standardization matching system of claim 4, wherein:
the standard symptom is divided into two or more and stored in the symptom table,
a corresponding code is assigned to each standard symptom,
a similar symptom determined to be a symptom similar to the standard symptom is previously set and matched to the code, and
the matching module sets a symptom expression for at least one symptom based on information received from the user terminal, matches a standard symptom corresponding to the set symptom expression, and checks code corresponding to the matched standard symptom.

6. The symptom standardization matching system of claim 5, wherein the similar symptom corresponds to that a predetermined number of pieces of information or more set as the symptom expression matched to the standard symptom are included in common with the standard symptom.

7. The symptom standardization matching system of claim 6, wherein the verification improvement module makes an additional question to the user terminal, adds and updates the symptom expression by taking into consideration an answer received from the user terminal, and sets the standard symptom or similar symptom again based on the updated information.

8. A service matching system for a symptom, comprising:
a user terminal;
a provider terminal configured to provide the user terminal with at least one related service corresponding to a symptom; and
a symptom standardization matching system of claim 1 connected to the user terminal and the provider terminal and configured to control the at least one related service.
